(19) United States
(12) Patent Application Publication
(10) Pub. No.: US 2016/0089260 A1
(43) Pub. Date: Mar. 31, 2016

(54) Method and Apparatus for Collecting Fluids that Drain From a Gastrostomy Tube

(71) Applicant: Marvin E. Hinds, Ashburn, GA (US)

(72) Inventor: Marvin E. Hinds, Ashburn, GA (US)

(21) Appl. No.: 14/497,459

(22) Filed: Sep. 26, 2014

(51) Int. Cl. A61F 5/449

(52) U.S. Cl. CPC A61F 5/449

(57) ABSTRACT

A method and apparatus including attachment of a fluid evacuation bag to the feeding port of the G-tube using an adapter fitting disposed between and interconnecting the G-tube and the evacuation bag. The adapter fitting has a proximal end that is sized to fit in and frictionally engage the feeding port of the G-tube, and a distal end that is sized to receive and frictionally engage tube end fitting extending from the evacuation bag. The adapter fitting allows open fluid communication from the G-tube to the evacuation bag so that fluids draining from the G-tube may be safely and conveniently collected and disposed of.

### BACKGROUND OF THE INVENTION

### Field of the Invention

The present invention relates to the field of medical devices, and more particularly to a method and apparatus for collecting fluids draining from a gastrostomy bag.

Gastrostomy tubes (G-tubes) are medical devices that allow nutrients and medications to be delivered directly to the stomach of a patient. G-tubes are inserted through incisions made in the abdomen and stomach and are secured in position by internal balloons and/or retention rings attached to the outer surface of the abdomen by adhesives or stitching.

G-tubes, however, may leak fluids onto the patient, caregivers, clothing, and bedding. Patients and caregivers are often exposed to embarrassing and offensive odors, and health hazards associated with contact with these fluids.

As a consequence of the foregoing situation, there has existed a longstanding need for a new and improved method and apparatus for collecting fluids that drain from a gastrostomy tube and the provision of such a method and apparatus is a stated objective of the present invention.

### BRIEF SUMMARY OF THE INVENTION

Briefly stated, the present invention provides a method and apparatus including attachment of a fluid evacuation bag to the feeding port of the G-tube using an adapter fitting disposed between and interconnecting the G-tube and the evacuation bag. The adapter fitting has a proximal end that is sized to fit in and frictionally engage the feeding port of the G-tube, and a distal end that is sized to receive and frictionally engage tube end fitting extending from the evacuation bag. The adapter fitting allows open fluid communication from the G-tube to the evacuation bag so that fluids draining from the G-tube may be safely and conveniently collected and disposed of.

### BRIEF DESCRIPTION OF THE SEVERAL VIEWS OF THE DRAWINGS

These and other attributes of the invention will become more clear upon a thorough study of the following description of the best mode for carrying out the invention, particularly when reviewed in conjunction with drawings, wherein:

FIG. 1 is a perspective view, showing the G-tube extending from the patient's abdomen, and being connected to an evacuation bag by the adapter fitting;

FIG. 2 is an exploded perspective view, illustrating the connection of the components used in the present invention;

FIG. 3 is a front view, showing the components connected;

FIG. 4 is a front view, showing a large size evacuation bag, suitable for use by a patient when taking an extended trip;

FIG. 5 is a front view, showing a medium size bag, suitable for short trips; and

FIG. 6 is a front view, showing a small size bag, suitable for use for a brief time.

### DETAILED DESCRIPTION OF THE INVENTION

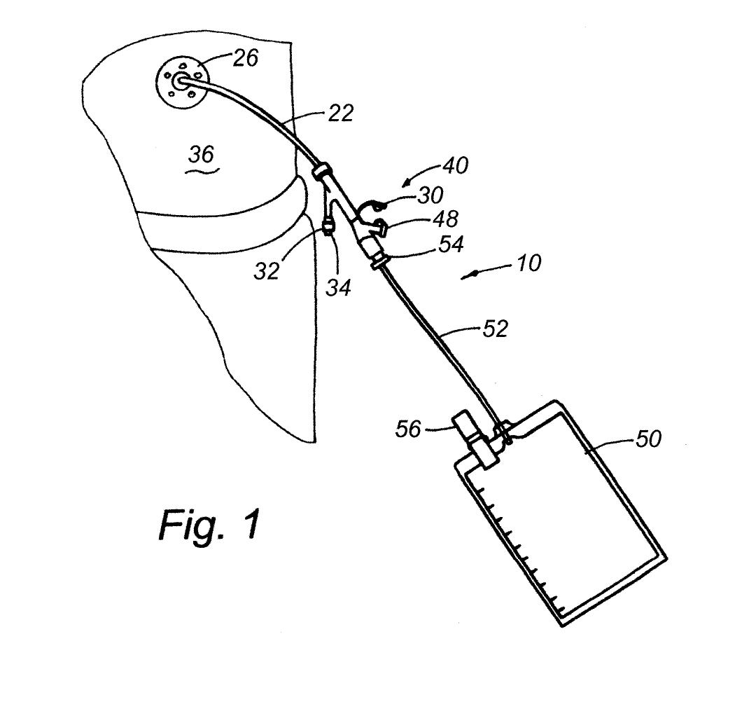

As can be seen by reference to the drawings, and in particular to FIG. 1, the medical device that forms the basis of the present invention is designated generally by the reference number 10. The medical device 10 includes a gastrostomy tube (G-tube) 20, an adapter fitting 40, and a fluid evacuation bag 50.

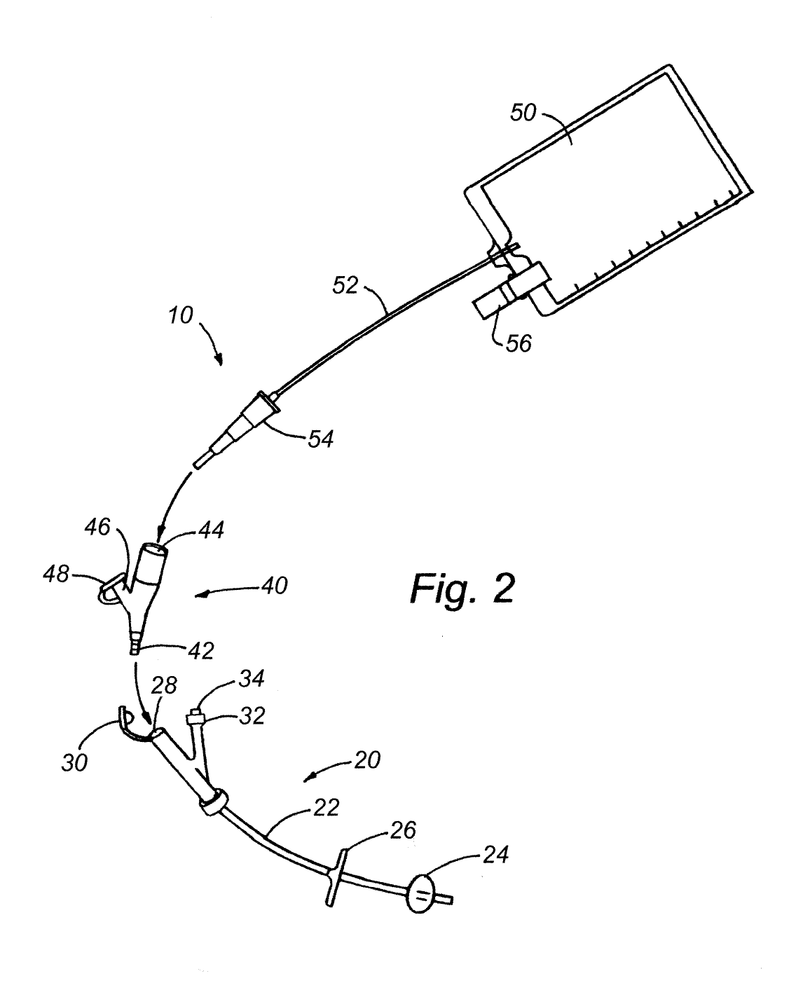

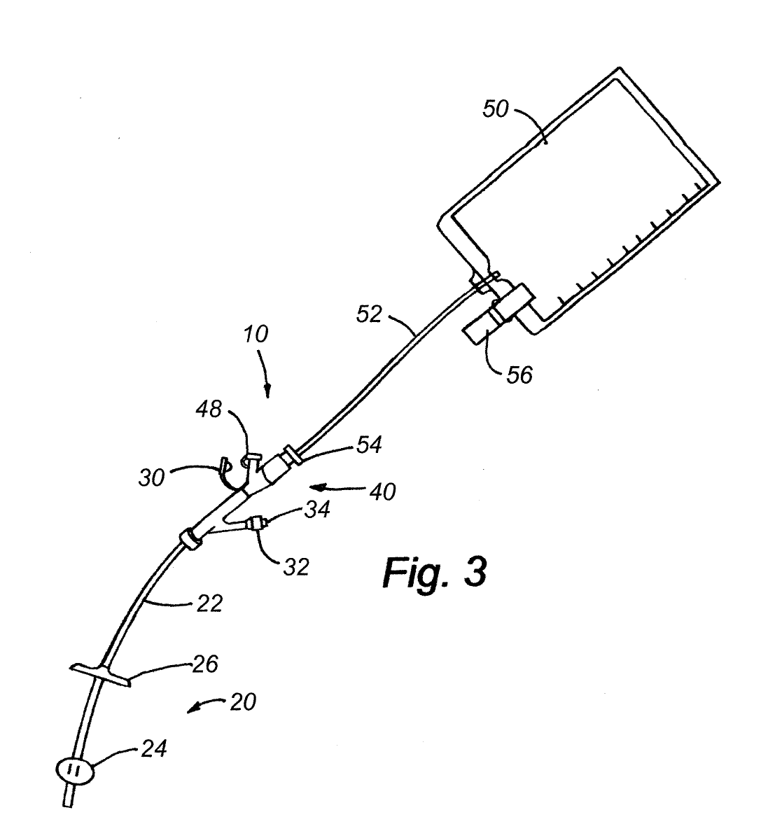

The G-tube 20 allows nutrients and medications to be delivered directly to the patient's stomach. As best shown in FIG. 2, the G-tube 20 illustrated is a conventional G-tube 20 that includes an elongated conduit 22 having a balloon 24 at the interior end, a retention ring 26 disposed out from the balloon 24, and a feeding port 28 with a port cover 30 at the exterior end. A balloon inflation port 32 with an inflation valve 34 is located near the exterior end. The G-tube 20 is inserted through incisions made in the abdomen 36 and stomach (not shown) of the patent where the balloon 24 is inflated inside the stomach and the retention ring 26 contacts the outer surface of the abdomen 36 and is secured by an adhesive or stitching.

The adapter fitting 40 includes a proximal end 42 sized to fit into and be frictionally engaged with the feeding port 28 of the G-tube 20, and an enlarged distal end 44. The adapter fitting also includes an offset auxiliary port 46 and port cover 48.

The fluid evacuation bag 50 has a tube 52 extending from the bag 50, and an enlarged tube end fitting 54. The end fitting 54 is sized to be received into and frictionally engaged with the enlarged distal end 44 of the adapter fitting 40. A clip 56 is attached to the bag 50 to secure the bag 50 to an article of clothing of the patient.

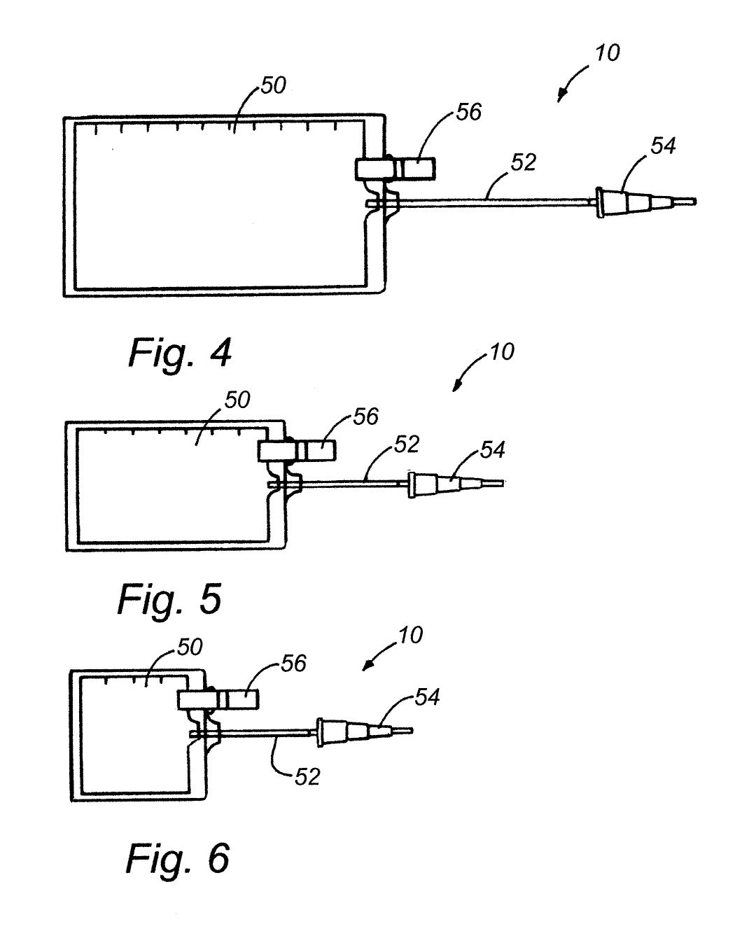

FIGS. 4-6 illustrate evacuation bags 50 of different sizes suitable for use over varying time intervals.

In use, the G-tube 20 is surgically attached to the patient to allow the patient to receive nutrients and medications directly into the stomach through the feeding port 28. When not in use for introducing materials into the stomach, the G-tube 20 may leak fluids that can cause odors and health hazards. To address this problem, the present invention provides for the attachment of fluid evacuation bag 50 to the G-tube 20 by means of the adapter fitting 40 to safely collect these fluids. At selected time intervals, or when the evacuation bag 50 is filled with fluids, the bag 50 is detached and discarded, to be replaced with an unused bag. This system minimizes embarrassing and offensive odors, and minimizes contact with fluids by the patient and caregivers to reduce health hazards.

Although only an exemplary embodiment of the invention has been described in detail above, those skilled in the art will readily appreciate that many modifications are possible without materially departing from the novel teachings and advantages of this invention. Accordingly, all such modifications are intended to be included within the scope of this invention as defined in the following claims.

Having thereby described the subject matter of the present invention, it should be apparent that many substitutions, modifications, and variations of the invention are possible in light of the above teachings. It is therefore to be understood that the invention as taught and described herein is only to be limited to the extent of the breadth and scope of the appended claims.

1. A method for collecting fluids draining from a gastrostomy tube that includes a feeding port, the method comprising the steps of:
providing an adapter fitting having a proximal end sized to fit into and frictionally engage the feeding port, and an enlarged distal end;
providing a fluid evacuation bag having a tube extending from the evacuation bag, and an enlarged tube end fitting;
frictionally attaching the proximal end of the adapter fitting to the gastrostomy tube at the feeding port;
frictionally attaching the enlarged distal end of the adapter fitting to the enlarged tube end fitting of the evacuation bag;
periodically detaching the evacuation bag from the adapter fitting after fluids have drained from the gastrostomy tube into the evacuation bag;
disposing of the detached evacuation bag; and
attaching an unused evacuation bag to the adapter fitting.

2. The method of claim 1, wherein the evacuation bag includes a clip for releasable attaching the evacuation bag to a clothing article of a patient.

3. The method of claim 1, wherein the adapter fitting includes an auxiliary port for introducing selected materials into the gastrostomy tube.

4. A medical device for collecting fluids draining from a gastrostomy tube that includes a feeding port, the device comprising:
an adapter fitting having a proximal end sized to fit into and frictionally engage the feeding port, and an enlarged distal end; and
a fluid evacuation bag having a tube extending from the evacuation bag, and an enlarged tube end fitting sized to fit into and frictionally engage the enlarged distal end of the adapter fitting.

5. The device of claim 4, further including a clip attached to the evacuation bag.

6. The device of claim 4, further including an auxiliary port formed in the adapter fitting between the proximal and distal ends.
